(19) United States
(12) Patent Application Publication
(10) Pub. No.: US 2026/0199077 A1
(43) Pub. Date: Jul. 16, 2026

(54) Plastically Deformable, Mechanically Strong, and Degradable Polymeric Airway Stents

(71) Applicant: Regents of the University of Minnesota, Minneapolis, MN (US)

(72) Inventors: Robroy MacIver, Minneapolis, MN (US); Marc Hillmyer, Minneapolis, MN (US); Arpan Biswas, Minneapolis, MN (US); Daniel Krajovic, Minneapolis, MN (US)

(21) Appl. No.: 19/449,072

(22) Filed: Jan. 14, 2026

Related U.S. Application Data
(60) Provisional application No. 63/745,164, filed on Jan. 14, 2025.

(51) Int. Cl. A61F 2/04; A61L 31/06; A61L 31/14

(52) U.S. Cl. CPC A61F 2/04; A61L 31/06; A61L 31/148; A61F 2002/043; A61F 2002/046; A61F 2210/0004; A61F 2250/001; A61F 2250/003

(57) ABSTRACT

Embodiments relate to stents for supporting an airway or other duct or plenum. The stents can be rapidly customized and inserted into the airway, obviating the need for subsequent intubation or for invasive procedures. The stent can be made of plastic or ductile materials, such that an expander can be positioned therein to cause the stent to plastically deform to provide a large passageway.

### CROSS-REFERENCE TO RELATED APPLICATIONS

This application claims priority to and the benefit of U.S. Provisional Application No. 63/745,164, filed 14 Jan. 2025, under 35 U.S.C. § 119 (e). The contents of the above-referenced provisional application are incorporated herein by reference in their entirety.

### STATEMENT REGARDING FEDERALLY SPONSORED RESEARCH

This invention was made with government support under CHE1901635 awarded by the National Science Foundation. The government has certain rights in the invention.

### BACKGROUND

The central airway system is a tube-like organ that connects the larynx to the lungs. It is responsible for the passage of inhaled and exhaled air. Stenosis, infection, trauma, congenital anomalies, and malignancies can damage the trachea and heavily influence mortality and morbidity among affected patients. Intervention with airway stents can keep the airway functional. Medical technologists have refined airway stents for more than a century, exploring various material classes, including metal, silicon, plastics, and their composites. Despite more than a century of development, 22% of implanted stents fail due to complications such as chronic granulation and erosion from the foreign material implanted, mucus plugging, and migration due to geometrical or mechanical property mismatching with the complex tracheobronchial anatomy of the individual patients.

Pediatric patients are more vulnerable to these complications, requiring additional interventions for stent removal and replacement. In addition, these stents inhibit the airway's normal secretory functions and require frequent chemical or mechanical pulmonary treatments to remain unobstructed. Recent innovations have addressed these issues, including the development of customized patient-specific bioresorbable airway stents using 3D or 4D printing techniques and the introduction of chiral auxetic engineered structures with ciliated epithelium to solve migration and mucus plugging. Outstanding problems associated with mechanical mismatch, including chronic granulation, arterial bronchial fistulas, and stent obstruction, must be solved.

One example of a condition that can be treated with airway stenting is tracheo-bronchomalacia (TBM). TBM is a condition where the trachea and/or bronchi collapse, commonly affecting children. This is due to the softness of their airways, which are prone to collapse. TBM can result in poorly ventilated regions of the lungs, leading to frequent respiratory infections and the need for positive pressure ventilation. As a result, children with TBM experience significant morbidity and mortality rates.

Conventional medical therapy for TBM involves chronic nebulizer treatments to break up mucous plugs formed by narrowed airways. Surgical treatment involves the use of stents to artificially keep the airways open. However, existing stents are not suitable for placement in the trachea or bronchus due to their design limitations. Silicon stents for adults have walls that are too thick, and if scaled down for use in children, they would obstruct the airway. Metal expandable stents, on the other hand, have a high in-growth rate, making their removal difficult or impossible.

As a result, there is a need for a treatment that can prevent airway collapse in children with TBM without the adverse effects of intubation or tracheal stents. The symptoms of TBM may improve over time as the trachea widens and becomes less pliable. However, caregivers of children with TBM face significant challenges in managing complex respiratory treatments, and if the child requires ventilation, they may need to leave their home during recovery. Therefore, it is crucial to develop an effective and safe treatment option that can alleviate the symptoms of TBM and improve the quality of life for both the affected children and their caregivers. Some existing solutions to these problems are described in U.S. Pat. No. 10,813,776, the contents of which are incorporated by reference in their entirety.

### SUMMARY

Embodiments described herein relate to a stent made of a special type of material known as a plastic body, meaning a body that exhibits permanent deformation of a solid without fracture by the temporary application of force. This stent runs along a central axis and has an initial central opening that also follows this axis. The stent is made of a block copolymer that can be plastically deformed, meaning it can be reshaped permanently under stress.

Additional features of this stent include the possibility of the block copolymer being bioresorbable, which means it can be absorbed by the body over time. The block copolymer can take the form of a tri-block structure or have more than three blocks, offering flexibility in its chemical structure. Specifically, this block copolymer can be an ABA-type, where the A-type component may be made of L-lactide, and in some cases, up to 90% (volume percentage) of this polymer is made up of this A-type component. Furthermore, the stent can be designed to degrade in stages within the body, with different portions degrading at different rates. These portions could vary in their material composition, thickness, or density and could be arranged in a specific pattern along the stent's axis.

The method for adjusting the size of this stent involves placing the stent within an airway and using an expander to widen its initial opening. This is achieved by inserting the expander into the stent's opening and then applying outward force, causing the stent to undergo plastic deformation and resulting in a larger aperture.

This sizing method also considers the same material properties as the stent itself, including the possibility of the block copolymer being bioresorbable and having a tri-block or more complex structure. It can also be an ABA-type block copolymer, predominantly made of L-lactide. The method can involve stents designed for multistage degradation, with different portions degrading at different rates based on their material properties or stent's design. These portions can be distinct in terms of composition, thickness, or density and arranged in patterns along the stent's axis.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1 and 2 depict a stent according to an embodiment herein, before and after expansion, respectively.

FIG. 3 is a chemical structure of an embodiment of a triblock copolymer usable in embodiment described herein.

FIG. 4 is a method flowchart according to an embodiment.

FIG. 5 is a set of graphs showing in vitro degradation study results, including: (a) total carbon content release and weight loss percentage over time; (b) size exclusion chromatography traces comparing pristine and degraded triblock copolymer showing molecular weight reduction; and (c) force versus displacement curves from uniaxial compression testing demonstrating mechanical property changes after degradation.

FIG. 6 is a set of analytical data supporting the degradation study, including: (a) a calibration curve for total organic carbon measurements showing linear fit parameters with equation, intercept, slope, and R-square value; (b) 1H-NMR spectra of degraded stent material showing end-group composition; and (c) differential scanning calorimetry thermogram showing thermal properties of degraded material.

FIG. 7 is a series of images depicting a cadaver deployment experiment in porcine lung tissue, showing: (a) the porcine trachea section and stent with balloon; (b) catheter-guided balloon insertion through the tracheal lumen; (c) balloon dilation; (d-f) post-deployment images showing the stent conforming to the tracheal lumen without buckling.

### DETAILED DESCRIPTION

Reference will now be made in detail to exemplary aspects of the present disclosure that are illustrated in the accompanying drawings. Wherever possible, the same reference numbers will be used throughout the drawings to refer to the same or like parts. Furthermore, when two similar embodiments are shown in different figures that have similar parts, those similar parts may be referred to using reference numbers iterated by a factor of 100. In such instances, the full description of those components may not be repeated, and it should be understood that they apply equally to the similar instances with similar reference numbers.

Described herein are mechanically robust and resilient airway stents that use biocompatible material such as PLLA-rich triblock copolymers, composed of poly(L-lactide) (PLLA) and poly(γ-methyl-ε-caprolactone) (PγMCL). These, among other plastic polymer materials, can be used for safely resizing the stent over time. Such airway stents feature tuned mechanical properties, biocompatibility, and biodegradability.

Copolymers with lower molecular weights exhibit Newtonian flow at high temperatures, while those with higher molecular weights demonstrated pronounced shear thinning when subjected to temperatures commonly used in some airway stent production methods, such as in 3D printing. Copolymers with intermediate molecular weights displayed the most favorable viscosity profiles for extrusion-based 3D printing. This enabled the fabrication of high-resolution solid and open-cell stents without significant sagging or delamination issues.

To deploy these stents effectively, we have devised a novel strategy involving radial dilation, which induced plastic deformation in the material, such as a triblock copolymer. Mechanical testing revealed that stents made from copolymers with intermediate molecular weights could deform plastically during the balloon dilation process, ensuring robust structural integrity after deployment. Stress-strain analysis demonstrated enhanced tensile strength and toughness in post-stretched samples, maintaining the stent's shape under expected physiological conditions.

In-vitro cytotoxicity assays using primary human airway epithelial cells were conducted to assess the compatibility of the stents over time. Encouragingly, the results indicated that stents made with these materials are biocompatible, paving the way for potential biomedical applications. Overall, these findings highlight the tremendous potential of triblock copolymers with intermediate molecular weights for fabricating 3D-printed airway stents. These stents offer superior mechanical performance, excellent printability, biocompatibility, and the added advantage of biodegradability. This discovery holds significant promise in revolutionizing airway stenting for improved patient outcomes.

Embodiments described herein include plastically deformable stents that can be expanded as needed throughout the lifetime of the stent. According to embodiments described herein, customized stents can be effectively and precisely deployed and are suitable for use in the small airways of children and infants. These stents can be deployed inside the airway, rather than using a “pexy” procedure in which a stent is attached to the outside of an airway, which improves airway collapse resistance. Furthermore, over time as the patient grows and the stent should be enlarged to correspond to the rest of their respiratory system, these stents can be expanded to a desired enlarged size without breaking or subsequently collapsing.

Although described herein with respect to airways (such as for treatment of tracheo-bronchomalacia (TBM) and the like) it should be understood that similar stenting could be performed for the vasculature or other passageways in the body.

### Definitions

As described in more detail below, a stent is made of a plastic polymer. Plasticity is referred to herein in its technical sense. Not all polymers are plastic. Rather, plasticity refers to a solid material capable of permanent deformation without fracture by the temporary application of force.

During manufacture, the plastic polymer may be viscoelastic. Viscoelasticity refers to the material's ability to exhibit both viscous (flow-like) and elastic (recovery) behavior when subjected to stress. Viscoelastic polymers have characteristics of both viscous fluids and elastic solids. They can undergo plastic deformation under moderate forces but can also recover their original shape to some extent when the stress is released.

The plastic nature of the stent material allows for its deformation during a sizing or resizing process. When the stent is deployed in an airway and an expander is positioned and expanded within the initial aperture, the viscoelastic body of the stent can undergo plastic deformation, allowing the initial aperture to transform into an expanded aperture. This plastic deformation is made possible by the viscoelastic properties of the stent material, such as in a block copolymer that can be plastically deformed.

Plastic deformation refers to a permanent change in the shape or size of a material when subjected to an external force. Unlike elastic deformation, where a material returns to its original shape after the force is removed, plastic deformation causes a lasting alteration in the material's structure. Plastic deformation typically occurs in materials such as polymers when the applied stress exceeds a certain threshold, known as the yield stress. Beyond this point, the material begins to permanently change shape without recovering its original form upon release of the force.

Polymers are described herein are block copolymers, tri-block copolymers, or various subcategories thereof. A block copolymer is a material made of two or more distinct polymer blocks that are chemically bonded together. These polymer blocks, each with its unique properties, combine to form a single macromolecule. The arrangement of the blocks can vary, with common structures being A-B, A-B-A, or A-B-C. The different blocks within the copolymer can exhibit diverse characteristics such as solubility, melting point, or mechanical strength.

ABA block copolymers are a type of copolymer consisting of two different kinds of polymer blocks arranged in a linear sequence. The name “ABA” refers to the arrangement of these blocks. The “A” blocks represent one type of polymer, while the “B” block represents a different type of polymer. The A and B blocks can be connected by covalent bonds. These copolymers can be formed through various polymerization techniques, such as living polymerization or controlled radical polymerization.

Biosorbability refers to the ability of a substance or material to be absorbed or taken up by biological systems, typically within the human body. It pertains to the capacity of a material to be broken down, metabolized, or eliminated through natural biological processes. Bioresorbable materials are designed to be compatible with the body, gradually degrading over time and eventually being assimilated or excreted without causing harm or leaving behind any unwanted elements thereof. This property is particularly valuable in the development of medical implants, such as sutures, stents, or drug delivery systems, where the material should provide temporary support before safely being absorbed by the body.

### Stent Design and Materials

Stents described herein are made of polymeric materials that have different beneficial characteristics that are not found in a single material. For example, the stents described herein can be bioresorbable, plastic, and resizable. In some embodiments, the stents described herein can have different degradation rates at different portions thereof, maintaining structural support while degrading at non-structural portions. Stent degradation can be accomplished by stent design. Portions of the stent with open cells may degrade faster than the solid portion because of the high surface area and less material in the open cell portion, for example.

The body of the stent can also be configured with a first portion and a second portion that degrade at different rates. This multi-stage degradation is achieved due to the viscoelastic properties of the material, which allow for controlled and differential degradation of the stent over time. Portions that degrade relatively more quickly can be dissolved or break off in small enough component parts to be cleared safely, such as by coughing or swallowing.

As one example, PLLA and poly (ε-caprolactone) (PCL) are widely used FDA-approved polymeric materials and can be used to form a block copolymer that provides advantages over conventional solutions. PLLA has a typical elastic modulus of 1-3 GPa, an order of magnitude higher than typical tissues, which could damage the tissues surrounding the implant. Further, during neck movement, the trachea may increase by 20% in adults and up to 46% in newborns. PLLA's low elongation at break and limited toughness restricts its applications in the biomedical field. In many ways, PLLA in isolation is therefore unsuitable for use in stents such as tracheal stents.

Block copolymers have great potential in the biomedical field because of their unique and tunable mechanical properties. Microphase separation in block polymers creates block domains that impart thermoplasticity to glassy-lean, elastomeric materials and toughness to glassy-rich, plastic counterparts. These domains may also influence the biological responses, which are similar in size to cell receptors and large proteins such as fibrinogen. Unfortunately, most polymeric biomedical devices and implants are derived from fossil fuels, producing unsustainable, linear plastic life cycles, motivating the investigation of renewable feedstocks. Block copolymers including PLLA and PγMCL were found to provide advantages over PLLA in isolation, while also being derivable from bio-based resources. Additionally, these block copolymers exhibit microphase separated structures, competitive mechanical and thermal properties, and are also biocompatible and biodegradable under prescribed conditions. PγMCL, an amorphous rubber, has a glass transition temperature (T_{g}) of −61° C. and a low entanglement molar mass (Mₑ) of 2.9 kg/mol, resulting in a densely entangled state even at modest molar masses. Incorporating PγMCL as a rubbery block in PLLA-b-PγMCL-b-PLLA (“LML”) triblock copolymers not only dramatically reduces the elastic modulus but also dramatically increases elongation at break.

Since its inception in the early 1980s, three-dimensional (3D) printing, also referred to as additive manufacturing (AM), has attracted considerable attention across numerous fields. Presently, 3D printing enables the fabrication of bespoke, intricate three-dimensional structures, previously challenging or even unfeasible to produce, with exceptional precision and accuracy. Within the biomedical sector, 3D printing offers myriad opportunities for creating prostheses, implants, drug delivery systems, and tissue regeneration scaffolds through the development of personalized devices tailored to individual patients. In many contexts, 3D printing-based manufacturing may also be more sustainable than conventional industrial manufacturing techniques in terms of CO₂ emissions and energy consumption. Therefore, a greener alternative to existing stents is contemplated that uses 3D printing in coordination with the triblock copolymer materials described above that are not derived from petroleum.

Among different commercially available 3D printing techniques, extrusion-based 3D printing is a widely recognized and commonly used method due to its low cost, low maintenance, and open-source nature. Because it does not rely on irreversible material curing, extrusion printing also permits structure recycling, offering a further sustainability advantage. Over the years, researchers have used extrusion-based 3D printing to develop tracheal grafts or implants. PLLA-rich LML triblock copolymers, which are suitable for extrusion-based 3D printing, can provide advantageous viscoelastic properties when molten for 3D printing for creating various solid and open-cell airway stents with excellent structural resolution. Furthermore, stents made of such materials can be deployed and later expanded using imposing radial dilation on cylindrical LML stents to induce permanent plastic deformation. Post-dilation characterization indicates that the stents are mechanically more resilient and capable of withstanding higher radial compression forces than commercially available silicon stents. A cytocompatibility assay with primary human airway epithelial cells demonstrates that the cells proliferate well on the surface of the triblocks over time and exhibit excellent viability. Thus, these PLLA-rich LMLs hold significant potential for developing next-generation customized stents and deploying them using plastic deformation in both airway and other luminal applications.

As described below, we have advanced the application scope of PLLA-rich LML triblock copolymers into the biomedical field. Targeting a slightly asymmetric triblock with 60% PLLA by volume produced triblocks with stiffness characteristics suited to address common issues with commercial silicone and metallic stents. By adjusting the overall molar mass at this block composition, we tailored the viscoelastic properties for efficient 3D printing, avoiding the filament layer sagging and poor interlayer welding, respectively, observed in the low- and high-molar mass limits, which enabled fabrication of high-resolution solid and open-cell stent structures. Mechanical testing revealed that the L14M16L14 triblock copolymer exhibited superior performance compared to conventional silicone and metal stents, particularly in terms of elongation at break, modulus, and radial load-bearing capacity. We leveraged the LML's plastic deformation to fix the stents in shapes required for tracheal implantation through a clinically accessible balloon dilation technique. The stents maintained structural integrity in the deformed state under compressive loads without buckling, ensuring reliability during tracheal application. Furthermore, in vitro cytocompatibility studies using primary human airway epithelial cells demonstrated excellent cell adhesion, proliferation, and viability on the surface of the 3D-printed LML structures. The high percentage of viable cells observed (approximately 92%) indicates that the L14M16L14 triblock copolymer not only supports cellular activity but also offers a biocompatible substrate conducive to tissue engineering applications. (The subscripts of L and M denote the block size in kg mol-1; hence, L14M16L14 is a triblock copolymer with a 16 kg mol-1 PγMCL mid-block and 14 kg mol-1 PLLA end-blocks.) This highlights the potential of the L14M16L14 triblock copolymer as a biomaterial for stent technology. Its mechanical properties and cytocompatibility make it a strong candidate for development and clinical use in tracheal and other medical interventions.

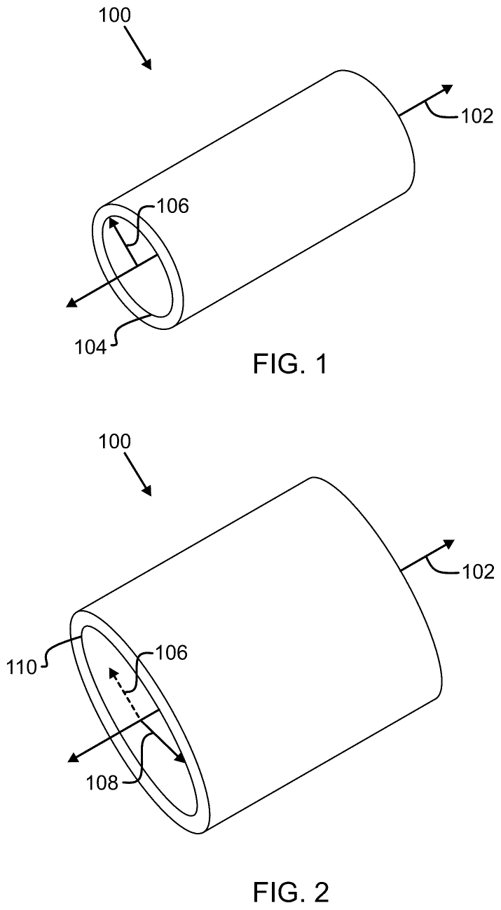

FIGS. 1 and 2 show an airway stent 100 according to one embodiment. Airway stents have conventionally been implanted via rigid or flexible bronchoscopy, which is standard in pulmonary medicine. However, both techniques have limitations, such as limited access to the upper lobes or distal airways for rigid bronchoscopy, and failure of stent expansion or miscalculated deployment for flexible bronchoscopy. A new deployment strategy is essential for overcoming the limitations of existing methods. The ability of PLLA-rich LMLs to deform plastically due to a low strain yield point could be utilized to create a stable and adjustable dilated structure of the stent during deployment.

Stent 100 as originally deployed extends along an axis 102 and defines an initial aperture 104 that also extends along the axis 102. The initial aperture 104 has an initial radius 106. Initial radius 106 may be sufficiently large for the airway or other lumen of a patient.

FIG. 2 shows the same stent 100, having been expanded by application of radial outward force on the initial aperture 104. For example, a balloon catheter can be positioned in the initial aperture 104 of the stent 100 and then expanded to provide the radial force. As a result of the application of radial outward force, the initial aperture 106 (shown in dashed lines in FIG. 2) is increased to an expanded radius 108 of an expanded aperture 110. As described in more detail below, stent 100 is made of materials that plastically deform from the initial aperture 104 of FIG. 1 to the expanded aperture 110 of FIG. 2 while maintaining sufficient post-expansion rigidity and strength to support surrounding structures (e.g., the tracheal wall).

Stent 100 of FIGS. 1 and 2 are simplified structures, essentially a tube segment. However, in alternative embodiments, different styles and structures can be used. For example, in one embodiment the stent 100 may be made of a mesh of triblock polymer, such that the stent 100 can be collapsed for implantation. Additionally, different portions of the stent 100 can be made of different density, thickness, or material composition than others. For example, bands extending in hoops orthogonal to the axis 102 could be embedded within the stent 100 that are thicker, denser, or material composition that are less biodegradable than the surrounding structure. In this way, after implantation or even expansion, the remaining portion of stent 100 can be dissolved or absorbed while the bands remain intact, providing structural support for the trachea.

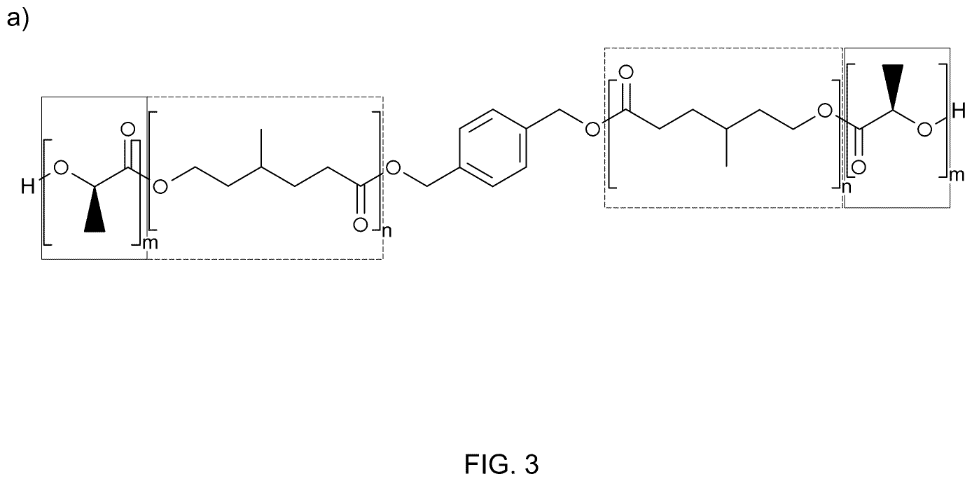

To create an appropriate PLLA-rich LML triblock copolymer, PLLA may be used as the end-block and PγMCL as the mid-block in a two-step ring opening transesterification polymerization protocol using 1,4-benzenedimethanol as the mid-block initiator. The chemical structure of the synthesized triblock copolymer is shown in FIG. 3. Here, as stated above, the LMLs with various molar masses have been designated as LpMqLp, where p and q stand for molar masses of PLLA and PγMCL blocks in kg mol-1. We targeted LML triblocks with a 60% PLLA volume fraction, recognizing that PLLA-rich triblocks exhibit a moderately high Young's modulus ranging from 1.1 GPa to ~900 MPa, yield stresses of 14 to 23 MPa, and elongations at break of nearly 600%. These mechanical properties are highly desirable for airway stents to prevent displacement and buckling under radial stress within the trachea, which can occur due to the low tensile modulus (10-20 MPa, as seen in silicone stents) or to avoid tissue damage or stenosis caused by very high tensile modulus (200 GPa, such as in metal stents).

The resulting triblock copolymers exhibit a desired rheological profile, including viscoelastic properties above melting temperature. Small-amplitude oscillatory shear (SAOS) measurements were carried out on LML melts to understand their flow properties both in the printhead nozzle and while welding to the previously deposited layers after extrusion. Complex viscosity (η*) is an important parameter to consider in extrusion-based 3D printing, providing information about the combined response of the elastic and the viscous components of the melt. The linear viscoelastic regime and complex viscosity of these materials at various temperatures (above melting temperature) may be tuned to exhibit Newtonian or non-Newtonian behavior in the temperature ranges expected for manufacture.

Extrusion-based 3D printing relies on depositing material layer by layer. A successful print requires structurally sound layers and good adhesion between the bed, the initial layer, and each subsequent layer. Various solid and open cell stent structures with 6 mm outer diameters and 5 mm inner diameters were prepared by extruding molten triblocks through a nozzle of 300 μm of extrusion-based 3D printer. The viable printing temperature increases with increasing the molar mass of the LML triblocks due to the commensurate increases in the viscosity and melting temperature. The low-molar mass L₁₁M₁₁L₁₁ generates less optimal structures during 3D printing. In contrast, the higher molar mass L₂₁M₂₂L₂₁ led to the slipping of the printed layers because of inadequate interlayer welding. LMLs with intermediate molar mass (L₁₆M₁₅L₁₆ and L₁₄M₁₆L₁₄) were found to allow the printing of high-resolution solid and open-cell stent structures without any sagging or delamination of the deposited layers.

Shear thinning behavior ensures a low viscosity value within the nozzle of a 3D printer, where polymers experience a high shear rate, enabling the extrusion step and lowering the required extrusion pressure. Additionally, stronger shear thinning allows a more rapid recovery of viscosity at the nozzle exit (low shear rate) that helps to retain the extruded shape, avoiding layer sagging. Hence, the less shear thinning for L₁₁M₁₁L₁₁ leads to sagging of the polymer in the printed structure, whereas higher shear thinning for L₁₆M₁₅L₁₆ or L₁₄M₁₆L₁₄ allows the construction of solid and open cell tubular structures with high-resolution, regular layers. Further, it was found that the complex viscosity at higher frequencies (100 rad s⁻¹) for L₂₁M₂₂L₂₁ (printing temperature range=220-230° C.) is higher than the complex viscosity suitable for 3D printing of polymers (10² Pa-s at 100 rads⁻¹ or 10³-10⁴ Pa-s at 0.1 rads⁻¹) which could be the reason for inadequate welding of the deposited layers for L₂₁M₂₂L₂₁. Moreover, L₂₁M₂₂L₂₁ thermally degraded during printing due to the high required temperatures as evident from size-exclusion chromatography traces collected before and after 3D printing. Therefore, it was discovered that LMLs with intermediate molar mass (L₁₆M₁₅L₁₆ or L₁₄M₁₆L₁₄) were best for printing high-quality, high-resolution open-cell stent structures, allowing the preparation of various open-cell airway stents with different engineered structures.

Small-(SAXS) and wide-angle X-ray scattering (WAXS) patterns collected for 3D printed filaments of L₁₄M₁₆L₁₄ showed no anisotropy resulting from PγMCL domain alignment or flow-induced crystallization, indicating no residual stress or memory of the extrusion flow in the finished stents. 3D printing also shows no effect on the melting temperature or crystallinity of L₁₄M₁₆L₁₄.

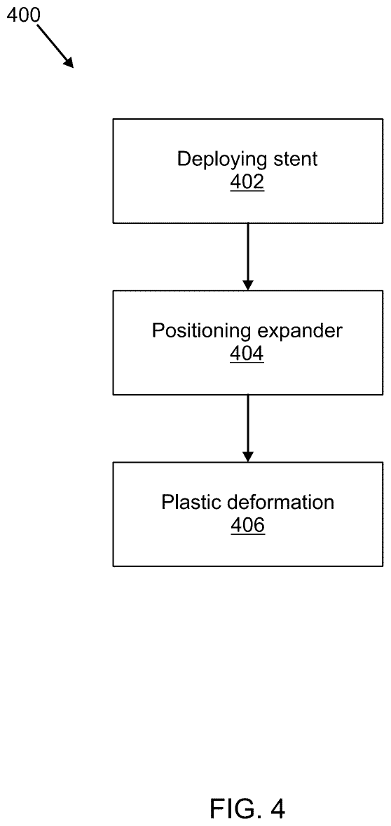

Expansion of the stent occurs as described in the flowchart of FIG. 4. As shown in FIG. 4, a method 400 includes three components. At 402, a stent is deployed. At 404, an expander (such as a balloon catheter or mechanical expander) is positioned within the stent that was deployed at 402. At 406, the expander that was positioned at 404 is used to provide radial outward pressure on the stent, causing plastic deformation.

Other optional elements of the method 400 can be included in other embodiments. For example, prior to deploying stent at 402, there may be a designing step that involves creating an appropriately sized or shaped stent for a particular patient. Additionally, there may be an optional step of 3D printing or other manufacturing process that takes advantage of the rheological features of the materials described above. After plastic deformation 406, there may also be a degradation step in which portions of the stent that was deployed at 402 decompose while leaving bands or other structure to support the surrounding lumen.

We devised a test to measure the post-yield shape retention ability of the triblocks. In this test, pristine dog-bone shaped samples of cold-crystallized (meaning quenched rapidly to room temperature after initial processing and subsequently reheated to ~100° C. for 5 minutes to encourage crystallization) triblock copolymers (initial length l₁) were stretched uniaxially to different stain percentages (length l₂) and held at a constant strain for 10 minutes to allow for stress relaxation in the deformed state. After removing the axial stress, the final length (l₃) was measured. We calculated the shape retention in the deformed state as

$${S}_{R} = \frac{{l}_{3} - {l}_{1}}{{l}_{2} - {l}_{1}} \times 100 \%$$

(i.e., when l₂=l₃, 100% of the deformed shape was retained). Shape retention increases from 54% for L₁₄M₁₆L₁₄ to 66% for L₂₁M₂₂L₂₁ and from 31% for 70% tensile strain to 66% for 100% tensile strain. Hence, S_{R} improved with increasing LML molar mass and with increasing tensile strain in the deformed state. We also re-assessed the tensile properties of the LMLs after the pre-stretching routine-hereafter denoted as “pre-stretched.” The pristine triblock samples pass through a yield point, after which they deform plastically. The tensile strength and elongation at the break of the pristine samples are 15±2 MPa and 432±35%, respectively. For pre-stretched samples, the tensile strength nearly doubled to 26.7±1 MPa, while the elongation at break fell to 282±25%. Notably, L₁₄M₁₆L₁₄ triblock is superior in terms of elongation at break compared to commercially available metal (45%) or silicone stents (120-170%). Interestingly, the overall tensile toughness calculated from stress-strain curve increased to 57±10 MJ m⁻³ in pre-stretched samples compared to pristine ones (47±10 MJ m⁻³). Therefore, the manufactured stent should be tougher after its deployment in the trachea through balloon dilation, contributing to the maintenance of structural integrity during neck movement. We also observed a decrease in Young's modulus from 667±45 MPa for pristine to 409±38 MPa in the pre-stretched samples. The tensile modulus of our stent material is lower than the commercially available metallic stents (200 GPa) and higher than that of typical silicone stents (10-20 MPa). Hence, our property-tailored LML stents counter common issues such as excessive granulation, tissue or tumor in-growth, arteriobronchiral fistulas caused by the high stiffness of metal stents, or migration and buckling for very soft silicone stents. The material's low strain yield point and flexibility in fabrication allow for the two key elements in our bioabsorbable airway stent design; these are controlled degradation and long-term support allowing for mucous clearance.

As described above, controlled degradation can be used to avoid emergent airway obstruction as the stent's mechanical properties degrade. This control is achieved by a multi-stage degradation. Supporting structures of the stent's combined ringed structure degrade sooner than the ring. Once the stent's ringed structure has grown into the airway, the supporting elements then degrade first. As the ring structures eventually degrade they then do so as small elements unable to obstruct the airway. The gaps between these long-term rings allow for mucous clearance and flexibility of the growing airway.

We employed in-situ tensile X-ray scattering to examine the deformation mechanisms of pristine and pre-stretched samples. PγMCL domain stretching accompanied the yielding transition, while subtle voiding occurred at higher strains. The lack of strong WAXS anisotropy suggests that the cold crystallized lamellae were largely unaffected by the initial yielding process. The elliptical SAXS feature in the low-strain regime of the pre-stretched specimen indicates some permanent stretching and alignment of the PγMCL domains, suggesting that alignment of PγMCL domains and the amorphous PLLA chains surrounding them was the preferential mechanism of plastic deformation in the pristine specimens. This alignment rationalizes the increase in tensile strength at higher strains resulting from pre-stretching.

The commercially available balloon dilation technique was insufficient in generating the necessary force to dilate the 3D-printed stents, attributed to the substantial wall thickness of approximately 1 mm present in the printed stents. Consequently, to validate the proposed deployment strategy—namely, the deployment of the fabricated stent through plastic deformation—solid stents featuring a diameter of 3.0 mm (l₁), a length of 23 mm, and a wall thickness of 0.1 mm were manufactured by rolling a flat rectangular sheet of L₁₄M₁₆L₁₄ and subsequently sealing the folded sheet through the application of heat to the joint. After fabrication, the stents were kept at 120° C. in an oven overnight to mimic the sterilization process, which resulted in cold crystallization of the stent. A catheter-guided balloon with an outer diameter of 8.0 mm (after dilation, l₂) was used to dilate the stent. The stent was dilated using a pressure gauge. The post-dilation diameter of the stent, measured after removing it from the balloon, is 5.0±0.3 mm (l₃). The shape retention

$$( {S}_{R} = \frac{{l}_{3} - {l}_{1}}{{l}_{2} - {l}_{1}} \times 100 \% )$$

of the stent was 67%, which is similar to the value measured using uniaxial stretching of the dog bone samples of L₁₄M₁₆L₁₄ triblock copolymer.

To operate effectively in the airway after deployment, a stent should preferably resist buckling under radial compression. Therefore, a series of solid stents with a 3 mm outer diameter, 10 mm length, and 0.1 mm wall thickness were fabricated and dilated with a balloon of 8 mm outer diameter (after dilation). The uniaxial compression test reveals that dilated airway stents have a load-bearing capacity of 0.9 N for 40% displacement of diameter, higher than the commercially available silicone stents (height 10 mm, diameter 16 mm, and wall thickness 1.2 mm) which exhibit a capacity less than 0.5 N for 40% displacement of diameter. Further, the L₁₄M₁₆L₁₄ resisted buckling during the uniaxial compression test.

### Cytocompatibility and Biodegradability of Triblock Copolymers

We investigated cell adhesion, proliferation, and viability on the triblock L₁₄M₁₆L₁₄ to motivate future biomedical applications. Cell adhesion to a material's surface directly reflects its suitability for tissue engineering. This adhesion gates critical processes such as proliferation and differentiation. Since epithelial cells line internal airways, we conducted in-vitro studies with primary human airway epithelial cells. These cells were seeded on the surface of 3D-printed flat rectangular triblock copolymer structures and then observed using a bright-field optical microscope. The control group consisted of cells grown on a well-plate surface. Both triblock and control groups showed spread and elongated cell morphologies, indicating good adhesion. To monitor cell proliferation on the triblock over time, we used the alamarBlue reduction assay at set intervals. The metabolic activity in cells converts resazurin into its highly fluorescent form. The reduction process, indicated by increased fluorescence intensity, corresponds directly to the number of actively respiring cells. Fluorescence intensity starts at 16% on day 1 and rises to 27%, 53%, and 70% on days 3, 7, and 14, respectively. This indicates that the metabolic activity of the primary epithelial cells increases over time, showing cells proliferating on the triblock surface.

Viability is an important parameter of the cells proliferating on the top of any material to determine the compatibility of the cells with the material. Therefore, we investigated the viability of the surface epithelial cells using a Live-dead assay, whereby we used calcine AM to stain the live cells and BOBO-3 Iodide to stain the dead cells only. Cell viability was determined using the density of the live cells with respect to the total live and dead cells. Typically, calcein AM, hydrolyzed by esterase in living cells, causes green fluorescence in their cytoplasm. Conversely, BOBO-3 Iodide binds to nucleic acids in dead cells, emitting red fluorescence. We found nearly 92% of cells viable on top of the 3D-printed LML on day 7, mirroring the viable cells in the control group. We concluded from these results that the L₁₄M₁₆L₁₄ is a cytocompatible substrate, allowing the primary airway epithelial cells to adhere and proliferate with good cell viability. Hence, the L₁₄M₁₆L₁₄ triblock copolymer is an excellent candidate as an implantable biomaterial.

Biodegradation is also an important property for any temporary airway implant to obviate surgical removal. We examined the in vitro degradation of the dilated stents (made of L₁₄M₁₆L₁₄) by immersing them in D-PBS solution and constantly agitating at 100 rpm at 37° C. For the degradation study, stents with 10 mm height, 3 mm outer diameter, and 100 mm wall thickness were dilated with a balloon of 8 mm outer diameter. The average final diameter and weight of the stents were 5±0.3 mm and 32.17±3.6 mg. The degradation was measured by determining the total carbon content of the D-PBS at predetermined time intervals. To investigate the total carbon content (TOC), 200 μL of aliquot was diluted with 780 ml of deionized water. The degradation of the dilated stents was expressed in terms of weight loss over time. The standard curve of weight vs total carbon content (TOC) was made by dissolving the polyethylene oxide in deionized water and measuring the total carbon content of solutions with different concentrations. We found no significant degradation of the dilated stents after 7 months.

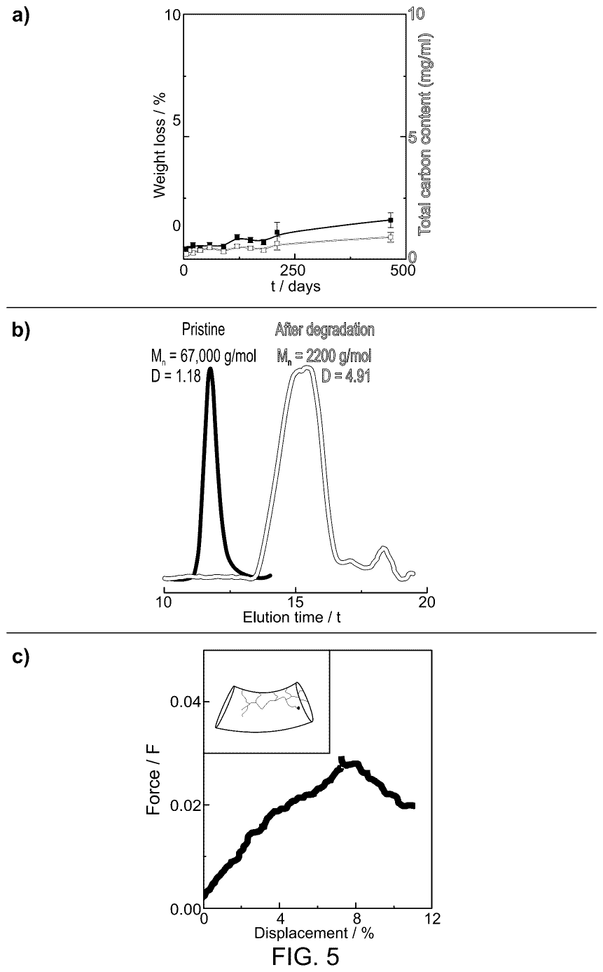

### In Vitro Degradation Study and Mechanical Property Evolution (FIG. 5)

To validate the multistage degradation characteristics of the L₁₄M₁₆L₁₄ triblock copolymer airway stent, an extensive in vitro degradation study was conducted over a 14-month period. As shown in FIG. 5(a), dilated stent samples were immersed in Dulbecco's phosphate-buffered saline (D-PBS) solution and maintained at 37° C. with constant agitation at 100 rpm to simulate physiological conditions. The degradation was monitored by measuring both the total organic carbon (TOC) content released into the D-PBS solution and the weight loss of the stent samples at predetermined time intervals. Unexpectedly and surprisingly, the results demonstrated only minimal mass loss of approximately 1.6% over the entire 14-month study period, with approximately 0.9 mg/mL of organic carbon released into the buffer solution. This limited mass loss despite extended exposure to aqueous conditions indicates that degraded polymer fragments remained largely trapped within the stent structure during the initial stages of degradation, a characteristic feature of the multistage degradation mechanism.

Size exclusion chromatography (SEC) analysis provided critical molecular-level insights into the degradation process. As illustrated in FIG. 5(b), the SEC traces reveal a dramatic shift in elution time between pristine and degraded samples. The pristine L₁₄M₁₆L₁₄ triblock copolymer exhibited a number-average molecular weight (Mn) of approximately 67,000 g/mol with a polydispersity index (D) of 1.18, indicating a well-controlled synthesis with relatively narrow molecular weight distribution. After 14 months of degradation in D-PBS, however, the molecular weight decreased substantially to approximately 2,200 g/mol, representing a reduction of approximately 97% from the pristine value. Concurrently, the polydispersity increased dramatically to 4.91, reflecting the generation of polymer chains with a broad range of molecular weights as hydrolytic chain scission proceeded throughout the material. The increase in elution time visible in the SEC trace confirms the decrease in hydrodynamic volume associated with this extensive molecular weight reduction. This disparity between the substantial molecular weight decrease and the minimal mass loss provides direct evidence of the multistage degradation behavior, wherein extensive chain scission occurs within the bulk material during initial stages without significant leaching of degraded fragments into the surrounding medium.

The mechanical consequences of this degradation are illustrated in FIG. 5(c) through uniaxial compression testing of both pristine and degraded dilated stents. Pristine dilated stents exhibited robust load-bearing capacity, withstanding forces of approximately 0.9 N at 40% strain displacement. This substantial mechanical strength ensures that the stent can resist the compressive forces present in diseased airways during the treatment period and maintain an open lumen to facilitate respiration. However, after 14 months of degradation, the load-bearing capacity of dilated stents was severely compromised, with the material exhibiting only approximately 0.03 N force capacity at 8% displacement before failure. This represents a reduction of approximately 97% in mechanical strength, demonstrating that the multistage degradation process had progressed sufficiently to eliminate the stent's structural support function. Visual inspection of the degraded samples, shown in the inset photograph of FIG. 5(c), reveals extensive surface cracking developed during the degradation process. These cracks provide visible evidence of the embrittlement associated with chain scission and loss of matrix entanglement, reflecting fundamental changes in material structure as polymer chains are cleaved into shorter fragments that can no longer effectively transfer stress across the material.

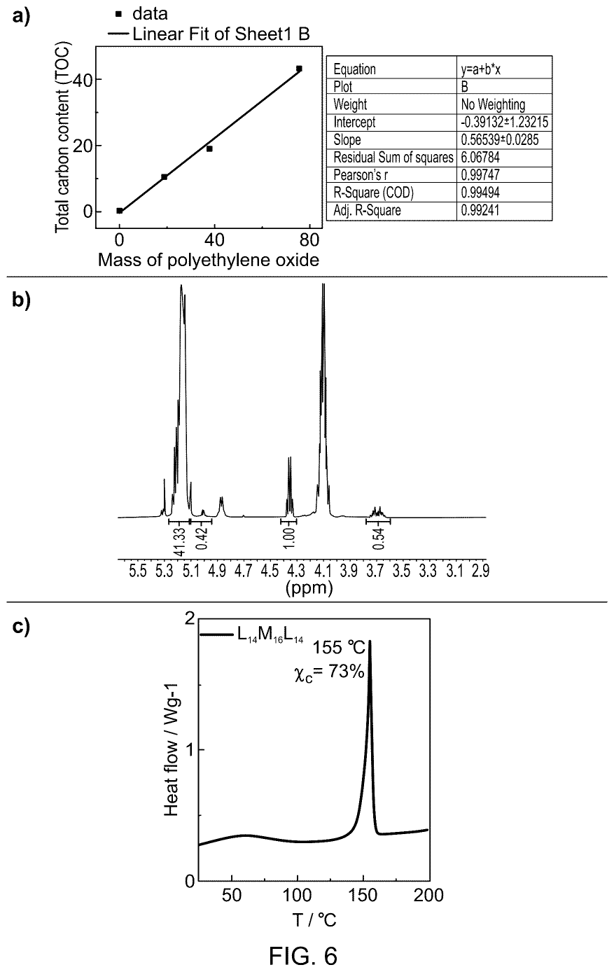

### Analytical Characterization of Degradation Mechanism (FIG. 6)

Comprehensive analytical characterization was performed to elucidate the molecular mechanisms underlying the multistage degradation behavior. Total organic carbon (TOC) analysis required careful calibration to ensure accurate quantification of polymer fragment release into the degradation medium. As shown in FIG. 6(a), a calibration curve was prepared by dissolving polyethylene oxide in deionized water at various concentrations and measuring the total carbon content of these standard solutions. The calibration data exhibit excellent linearity, as evidenced by the fit equation y=a+bx with the following parameters: Intercept (a)=−0.39132±1.23215, Slope (b)=0.56539±0.0285, Residual Sum of Squares=5.06784, Pearson's r=0.99747, R-Square (COD)=0.99494, and Adjusted R-Square=0.99241. The high Pearson correlation coefficient and R-square value confirm the quality and reliability of this linear relationship, providing confidence in the TOC measurements used to quantify organic carbon release during degradation.

Proton nuclear magnetic resonance (1H-NMR) spectroscopy of degraded stent samples provided critical insights into the chain scission mechanism. The 1H-NMR spectra shown in FIG. 6(b) reveal that after 14 months of degradation, the molar mass of the PLLA arms was reduced from approximately 14 kg/mol in pristine material to approximately 3.1 kg/mol, indicating considerable chain scission within the PLLA blocks. Significantly, the PγMCL end-group signals (CH₂) became visible in the degraded sample and integrate at a ratio of 0.54:1 with respect to the PLLA end-group signals. Because the PLLA end-group contains only one proton while the PγMCL end-group contains two protons, this corresponds to an end-group abundance ratio of 0.27:1, meaning that approximately 79% of the exposed chain ends belong to PLLA and approximately 21% belong to PγMCL. This ratio agrees remarkably well with the 73% weight fraction of PLLA in the triblock copolymer composition, providing strong evidence that chain scissions occur essentially randomly throughout both the PLLA and PγMCL blocks without preferential degradation of either component. This random chain scission mechanism is a fundamental characteristic of the multistage degradation process and ensures predictable breakdown behavior.

Differential scanning calorimetry (DSC) provided complementary information about the thermal properties and crystalline structure of degraded material. The DSC thermogram presented in FIG. 6(c) shows the first heating scan of degraded stent material, which is largely featureless except for a prominent melting peak at 155° C. The melting enthalpy (ΔH) measured from this peak is 50.195 J/g, corresponding to a crystallinity (Xc) of approximately 73%. This represents a substantial increase from the approximately 40% crystallinity observed in pristine samples. The decrease in melting temperature from approximately 166° C. in pristine material to 155° C. in degraded samples, coupled with the increased crystallinity, is consistent with the formation of shorter PLLA chain fragments that assemble into thinner crystal lamellae. These short chain fragments possess enhanced molecular mobility compared to the longer chains in pristine material, allowing them to reorganize and crystallize more readily. The featureless nature of the thermogram aside from the melting peak supports the conclusion that the degraded material is composed of short, highly mobile chains that have assembled a large weight fraction of thin crystal lamellae, corroborating the extensive chain scission evidenced by SEC and NMR data and representing an intermediate stage in the multistage degradation profile.

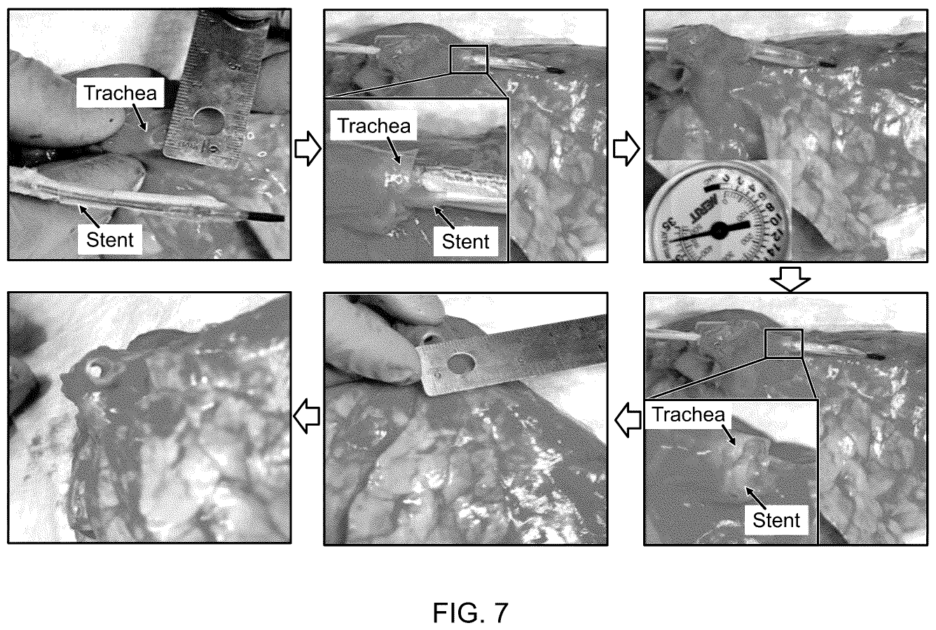

### Cadaver Deployment Validation (FIG. 7)

Clinical feasibility of the balloon-induced plastic deformation deployment strategy was validated through cadaver studies using porcine lung tissue, which provides anatomical features representative of human airways. As shown in FIG. 7(a), a section of porcine trachea with an inner diameter of approximately 6 mm was selected for this experiment. A stent with an outer diameter of 4 mm and wall thickness of 0.3 mm was fabricated as previously described. A catheter-guided balloon with an outer diameter of 7 mm after full dilation was inserted into the lumen of the stent to enable controlled expansion through plastic deformation. This dimensional configuration—with the balloon outer diameter exceeding the target deployment diameter—ensures sufficient radial pressure can be applied to induce permanent plastic deformation of the L₁₄M₁₆L₁₄ triblock copolymer material.

The deployment procedure is illustrated in FIGS. 7(b) and 7(c). The catheter-guided balloon was inserted through the hollow lumen of the porcine trachea to position the stent appropriately at the desired location, as shown in the zoomed image of FIG. 7(b). The positioning of the balloon and stent assembly within the tracheal lumen prior to expansion is visible, demonstrating the minimally invasive nature of the catheter-based delivery approach. The balloon was then dilated by applying a controlled pressure of 3.3 MPa using a pressure gauge, as depicted in FIG. 7(c). This applied pressure exceeded the yield strength of the L₁₄M₁₆L₁₄ triblock copolymer material, causing permanent radial expansion of the stent from its initial 4 mm outer diameter to match the 6 mm inner diameter of the porcine airway lumen through plastic deformation. Following stent dilation at this pressure, the balloon was deflated and removed, leaving the plastically deformed stent in place within the trachea to provide structural support.

Post-deployment imaging provided critical evidence of stent deployment and conformational fit to the airway anatomy. As illustrated in FIGS. 7(d)-7(f), the stent matches the lumen's diameter following deployment, with the final stent diameter measuring 6 mm as shown in FIG. 7(e). As demonstrated in FIG. 7(f), the stent was successfully deployed without any buckling or geometric distortion despite the substantial plastic deformation required to expand from 4 mm to 6 mm diameter. The stent conformed seamlessly to the shape of the tracheal lumen and maintained its expanded configuration due to the permanent plastic deformation of the triblock copolymer material. This conformal fit to the airway anatomy represents a significant advantage over conventional fixed-diameter stents, which often suffer from geometric mismatch problems that can lead to migration or tissue trauma. The successful cadaver deployment directly validates the clinical feasibility of deploying LML stents in the trachea and other human luminal organs using the balloon expansion technique described herein, demonstrating that the plastic deformability of the L₁₄M₁₆L₁₄ triblock copolymer enables sizing to individual patient anatomy while maintaining sufficient mechanical strength to resist airway collapse.

In embodiments, a kit or package can be provided to a medical professional for introducing a stent such as those described above. For example, the kit could include a quantity of material for use in an additive manufacturing process. The kit could also include a guide wire and driving catheter, in embodiments, or other components that would be used to determine the size and shape of a patient's airway.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A stent comprising a plastic body extending along an axis and defining an initial aperture, the initial aperture also extending along the axis, the plastic body made of a plastically deformable block copolymer, the plastic body being capable of permanent deformation without fracture by the temporary application of radial force.

2. The stent of claim 1, wherein the plastically deformable block copolymer is bioresorbable.

3. The stent of claim 1, wherein the plastically deformable block copolymer is a tri-block copolymer.

4. The stent of claim 1, wherein the plastically deformable block copolymer is made of more than three blocks.

5. The stent of claim 1, wherein the deformable block copolymer is an ABA-type block copolymer.

6. The stent of claim 5, wherein the A-type components of the ABA-type block copolymer are L-lactides.

7. The stent of claim 5, wherein at least 60% of the ABA-type block copolymer is the A-type component.

8. The stent of claim 1, wherein the plastic body is configured for multi-stage degradation by including:
a first portion that degrades in the body at a first rate; and
a second portion that degrades in the body at a second rate.

9. The stent of claim 8, wherein the first portion differs from the second portion in at least one of the group consisting of: material composition, material thickness, and material density.

10. The stent of claim 8, wherein the first portion and the second portion are arranged in a pattern along the axis of the plastic body.

11. A method for sizing a stent in vivo, the method comprising:
deploying a stent in an airway, the stent comprising a plastic body extending along an axis and defining an initial aperture, the initial aperture also extending along the axis, the plastic body made of a plastically deformable block copolymer;
positioning an expander in the initial aperture;
expanding the expander to apply a radially outward force against the plastic body, thereby causing plastic deformation of the stent such that the initial aperture becomes an expanded aperture.

12. The method of claim 11, wherein the plastically deformable block copolymer is bioresorbable.

13. The method of claim 11, wherein the plastically deformable block copolymer is a tri-block copolymer.

14. The method of claim 11, wherein the plastically deformable block copolymer is made of more than three blocks.

15. The method of claim 11, wherein the deformable block copolymer is an ABA-type block copolymer.

16. The method of claim 15, wherein the A-type components of the ABA-type block copolymer are L-lactides.

17. The method of claim 15, wherein at least 90% of the ABA-type block copolymer is the A-type component.

18. The method of claim 11, wherein the plastic body is configured for multi-stage degradation by including:
a first portion that degrades in the body at a first rate; and
a second portion that degrades in the body at a second rate.

19. The method of claim 18, wherein the first portion differs from the second portion in at least one of the group consisting of: material composition, material thickness, and material density.

20. The method of claim 18, wherein the first portion and the second portion are arranged in a pattern along the axis of the plastic body.
